(19) United States
(12) Patent Application Publication
(10) Pub. No.: US 2007/0260230 A1
(43) Pub. Date: Nov. 8, 2007

(54) Opto-mechanical Apparatus and Method for Dermatological Treatment

(75) Inventors: David W. Youngquist, San Jose, CA (US); George Frangineas, Fremont, CA (US); Thomas R. Myers, Palo Alto, CA (US)

Correspondence Address: RELIANT / FENWICK;c/o FENWICK & WEST, LLP, 801 CALIFORNIA STREET, MOUNTAIN VIEW, CA, 94041, US

(73) Assignee: RELIANT TECHNOLOGIES, INC., Mountain View, CA (US)

(21) Appl. No.: 11/744,161

(22) Filed: May 3, 2007

Related U.S. Application Data
(60) Provisional application No. 60/798,012, filed on May 4, 2006.

(51) Int. Cl. A61B 18/18

(52) U.S. Cl. 606/9

(57) ABSTRACT

An imaging sensor forms an image of at least a portion of a moving element that moves relative to a dermatological treatment handpiece in response to motion of the handpiece across the skin. A processor uses multiple images from the imaging sensor to determine changes in position or velocity of the moving element. The treatment energy source is adjusted or triggered in response to the calculation. Image relaying optics may be used to remotely position the imaging sensor away from the handpiece.

### CROSS-REFERENCE TO RELATED APPLICATION

This application claims priority under 35 U.S.C. § 119(e) to U.S. Provisional Patent Application Ser. No. 60/798,012, “Opto-mechanical Apparatus and Method for Dermatological Treatment,” filed May 4, 2006.

### BACKGROUND OF THE INVENTION

1. Field of the Invention

This invention relates generally to apparatus and method for treatment using an optomechanical imaging sensor to measure one or more positional parameters of the energy delivery handpiece. More particularly, it relates to imaging of a moving element to provide relative positional feedback for a cosmetic dermatologic treatment handpiece.

2. Description of the Related Art

Cosmetic and non-cosmetic dermatological treatments are commonly performed with lasers, bipolar and monopolar radio-frequency (RF) sources, RF plasma sources, LEDs, and flashlamp systems. One limitation of these treatments is the controlled delivery of energy to the treatment sites. In most systems, the energy treatment handpiece is fired and then moved by the operator to a new location where it is stopped and fired again. This type of system is slow because it requires precise placement of the handpiece for firing each energy pulse and it is subject to stitching errors where the placement of the handpiece is not precise. Stitching errors can cause over- or under-treatment.

One way to increase treatment speed and reduce stitching errors is to move the handpiece continuously over the skin within a desired treatment region while automatically firing the treatment source at a desired time. In some systems, this technique has been implemented by firing the energy source at a preselected pulse repetition rate while the user moves the handpiece at a preselected speed across the skin. If the user does not move at the appropriate rate, the desired energy dose is not delivered and over- or under-treatment can result. There is a need for a system where the delivered treatment dose is controlled in response to changes in motion of a continuously moving handpiece across the skin.

Control systems have been developed that provide feedback related to the movement of the handpiece across the skin. These systems are complicated, costly, or limited in effectiveness. For example, Weckwerth et al (U.S. Pat. No. 6,758,845) describes a system in which uniformly spaced indicia are marked onto a target skin and a laser treatment system is automatically triggered in response to detected movement of the handpiece across the indicia. This system is complicated in that it requires that the skin be marked with uniformly spaced indicia. It is difficult to place uniformly spaced indicia very closely together. Indicia that are not closely spaced do not provide good resolution for measurement of handpiece position. In addition, the suggested indicia are drawn using a visible coloring that would preferably be removed following treatment. Removal of the indicia can be a time consuming additional step and thus reduces the desirability of implementing this approach.

Talpalriu et al (U.S. Pat. No. 6,171,302) describes a system that uses movement related feedback. In one embodiment, for example, Talpalriu describes a rotating element that rotates due to contact with the skin and whereby the rotating element has evenly spaced reflective and nonreflective elements that are detected by a non-imaging sensor. However, this implementation requires finely spaced elements in order to obtain fine resolution. The Talpalriu system may be inaccurate when used with gels or anesthesias, which are commonly used in dermatological treatments. In addition, the Talpalriu system will not be able to distinguish the direction of the movement, which can be important in the case where short back and forth motions (e.g. motion from a shaky hand) could cause overtreatment if the direction of motion is not detected.

There is a need for an apparatus and method that control the treatment dosage of a moving handpiece in response to changes in a handpiece positional parameter, such as handpiece position, speed, or velocity. There is a further need for this apparatus and method to provide fine resolution for measurement of a handpiece positional parameter and to be inexpensive and simple to implement without the need for finely spaced indicia placed on the skin and for the apparatus and method to distinguish between handpiece motion in two opposite directions and/or in cross directions.

### SUMMARY OF THE INVENTION

The present invention overcomes the limitations of the prior art by using an imaging sensor to detect motion of a moving element in a dermatological treatment handpiece. A handpiece is configured to receive energy from an energy source and to deliver the energy to a skin for dermatological treatment. A moving element moves in response to the motion of the handpiece across the skin and that motion is detected by an imaging sensor that captures multiple images of at least a portion of the moving element. A processor compares at least two of the multiple images to determine at least one positional parameter of the moving element. A controller controls the energy source to alter the dermatological treatment in response to the processor's determination.

In one aspect of the invention, the processor can distinguish between motions of the moving element in two opposite directions relative to the orientation of the handpiece. In some embodiments, the processor can distinguish between motion of the moving element in two cross (e.g., perpendicular) directions relative to the orientation of the handpiece.

The treatment energy source may emit electromagnetic energy or ultrasonic energy. For example, the energy source may be a laser source, a flashlamp, an LED source, a radio-frequency (RF) source, a radio-frequency source that delivers energy to plasma for treatment of the skin, or an ultrasonic transmitter. Each of these sources may operate in continuous wave (CW) mode or in pulsed mode. Each of these sources may comprise multiple source elements.

The apparatus set forth above can further comprise image relaying optics that form parts of the optical path between at least a portion of the moving element and the imaging sensor. In a preferred embodiment, the image relaying optics is a fiber array.

In various embodiments, the controller can have different responses to the motion of the moving element. For example, in one embodiment, the controller automatically triggers the energy source in response to a measured motion of the moving element of a predetermined distance.

In another embodiment, the controller adjusts the firing rate of the energy source to a nonzero firing rate in response to the determined motion.

In another embodiment, the controller adjusts the power level of the energy source in response to a change in at least one of speed and velocity of the moving element.

In yet another embodiment, the controller adjusts the pulse repetition rate in response to the determined motion.

In yet another embodiment, the controller adjusts the energy dose in response to the determined motion.

The imaging sensor may comprise a CCD chip or CMOS detector array that is attached to the handpiece or located remotely. In one embodiment, the CCD chip or CMOS detector array is located at least 50 centimeters from the handpiece.

The moving element can be a rotating element. The shape of the moving element can be substantially spherical, substantially a round or polygonal cylinder, or may be not round. The moving element may comprise a band. Any of the above described moving elements may further comprise a textured surface that enhances the traction of the moving element on the skin or a patterned image. The patterned image may be repeating or non-repeating. A repeating image preferably comprises at least two different elements and/or two different spacings between adjacent elements. The moving element may comprise a single-piece or may be a segmented moving element. Two or more moving elements may be used.

In a preferred embodiment, discrete treatment zones are created in the target region of skin. In a preferred embodiment, a scanner can be used to direct energy from the treatment energy source to different portions of the target region. In some embodiments, a patterning element can direct energy from the treatment energy source to multiple discrete portions of the target region.

In a preferred embodiment, multiple images are captured within 50 milliseconds (ms), and more preferably within 5 ms in order to obtain accurate measurements with high resolution for a positional parameter of the moving element.

In another preferred embodiment, visible, auditory, or vibratory feedback is provided to the user to indicate that the handpiece velocity is outside (or inside) a desired range.

Other aspects of the invention include methods corresponding to the devices and systems described above.

### BRIEF DESCRIPTION OF THE DRAWINGS

The invention has other advantages and features which will be more readily apparent from the following detailed description of the invention and the appended claims, when taken in conjunction with the accompanying drawings, in which

FIG. 1 is a side view of an embodiment of the invention.

FIGS. 2A-2C are side views that illustrate different embodiments of a moving element according to the invention.

FIG. 3 is a side view of an embodiment of the invention that uses an imaging waveguide array.

FIGS. 4A-4G illustrate example patterns for the moving element.

FIG. 5 illustrates an embodiment of the invention that includes two moving elements and two imaging systems.

FIGS. 6A-6B are front views of embodiments of the removable tip aspect of the invention.

### DETAILED DESCRIPTION OF THE PREFERRED EMBODIMENTS

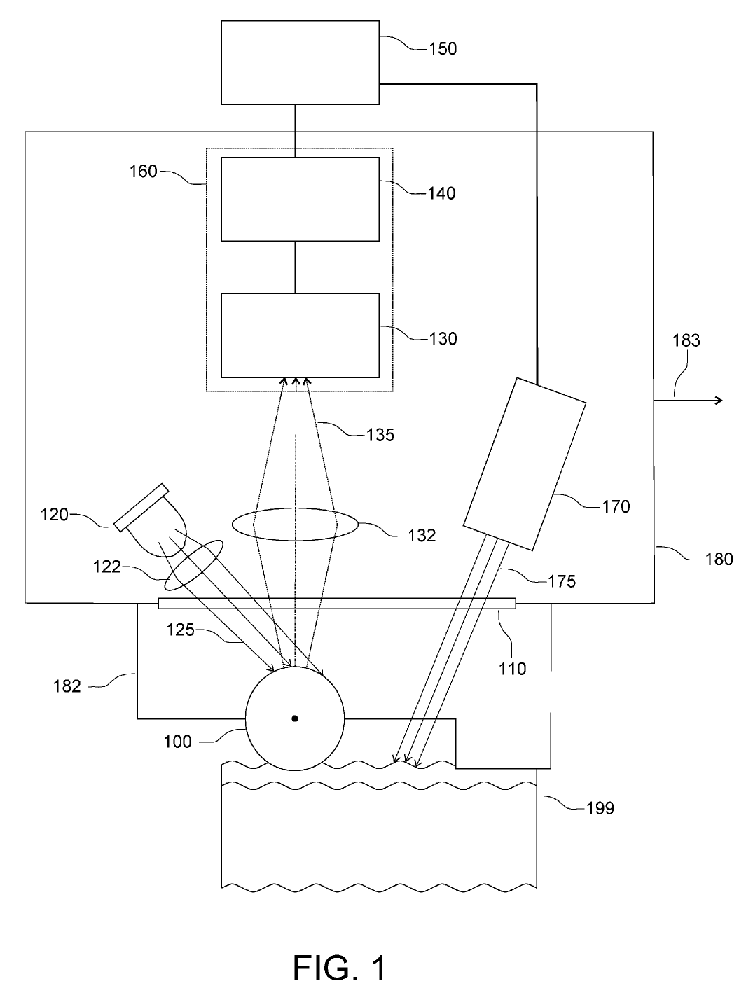

There is a need for an apparatus and method that control the treatment dosage of a moving handpiece in response to changes in a positional parameter of the handpiece. For purposes of this application, positional parameter means positional measurement results such as position, speed, velocity, acceleration, or orientation. Positional parameters can be either absolute or relative. FIG. 1 illustrates a side view of one embodiment of the invention that addresses this and other needs. In this embodiment, a handpiece 180 contains a treatment energy source 170 that is directed to provide treatment energy 175 to a target region of skin 199 to cause a dermatological effect in the skin 199. The handpiece 180 further comprises a removable tip 182 that is detachable from the rest of the handpiece 180. The removable tip 182 comprises a moving element 100 that moves relative to the rest of the handpiece 182 in response to movement of the handpiece 182 across the skin 199. The motion of the moving element 100 is detected by an imaging sensor 130 that detects imaging light 135 from the moving element 100 using an imaging lens 132. To increase the level of the imaging light 135, illumination 125 from an illumination source 120 can be directed through an imaging lens 122 towards the moving element 100. The imaging sensor 130 is configured to deliver images to a sensor analysis processor 140, which is connected to a controller 150 that controls the treatment energy source 170. An optional transparent window 110 may be used to protect optics inside the handpiece. The handpiece is manually moved along a direction 183. This motion can be forwards or backwards (i.e. the direction 183 can be reversed).

The moving element 100 can move in response to friction between the moving element 100 and the skin 199. At different times, the imaging sensor 130 detects multiple images from at least a portion of the moving element 100. The sensor analysis processor 140 evaluates multiple images from the imaging sensor 130 to determine at least one positional parameter of the moving element 100. In one embodiment, the processor 140 does so by making calculations based on the images from imaging sensor 130. The controller 150 uses the results from the sensor analysis processor 140 to alter the output of the treatment energy source 170 in a manner that affects the dermatological treatment.

In some embodiments, the controller 150 can adjust parameters of the treatment energy source 170 in proportion to the change in velocity in order to deliver a uniform dose to the skin. Examples of parameters of the treatment energy source 170 that can be adjusted by the controller 150 are power level, pulse repetition rate, pulse timing, and pulse duration. In other embodiments, the controller 150 can adjust parameters of the treatment energy source 170 such that the overall treatment response is less dependent on the motion of the handpiece than it would be if the dose were uniform, such as in the case where the effects of bulk heating of the tissue are substantial and are substantially affected by changes in handpiece speed or velocity. In yet other embodiments, the controller 150 automatically triggers the energy source in response to a measured movement of the moving element 100 of a predetermined amount.

The controller 150 may be a computer or any other control system suitable for adjusting the parameters of the treatment energy source 170 in such a way as to affect the dermatological treatment. For purposes of this application, “adjusting parameters” of the treatment energy source does not include actions that have the sole effect of stopping treatment. “Adjusting treatment,” however, may include stopping treatment as part of an adjustment range or as a selected response to a particular measurement.

The moving element 100 may further comprise a patterned image at least a portion of which is imaged by the imaging sensor 130 and can enhance the response of the imaging sensor 130, particularly in low light conditions or in conditions where there is topical ointment applied to the surface of the skin during treatment.

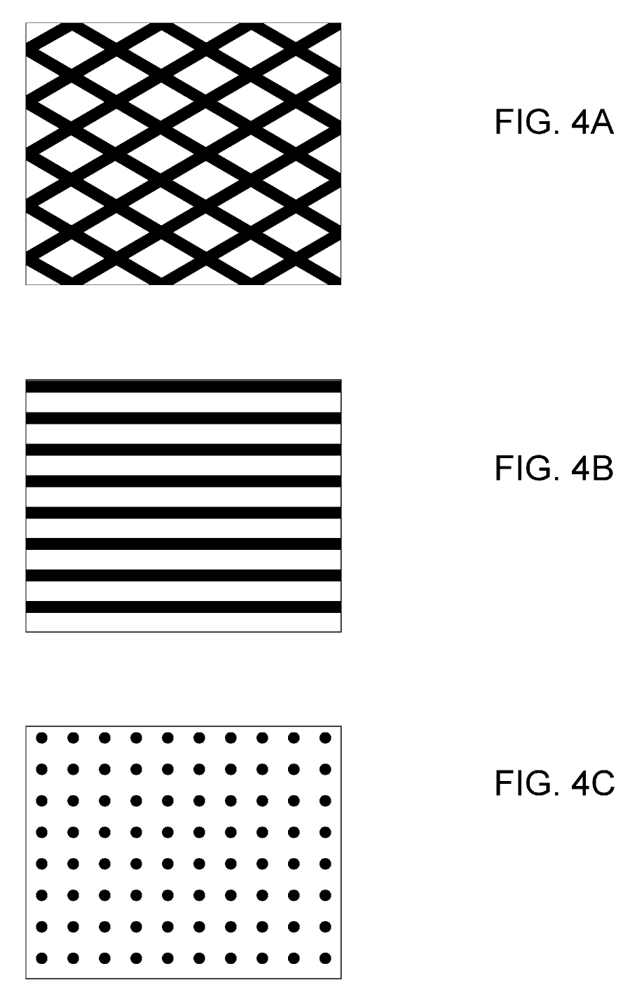

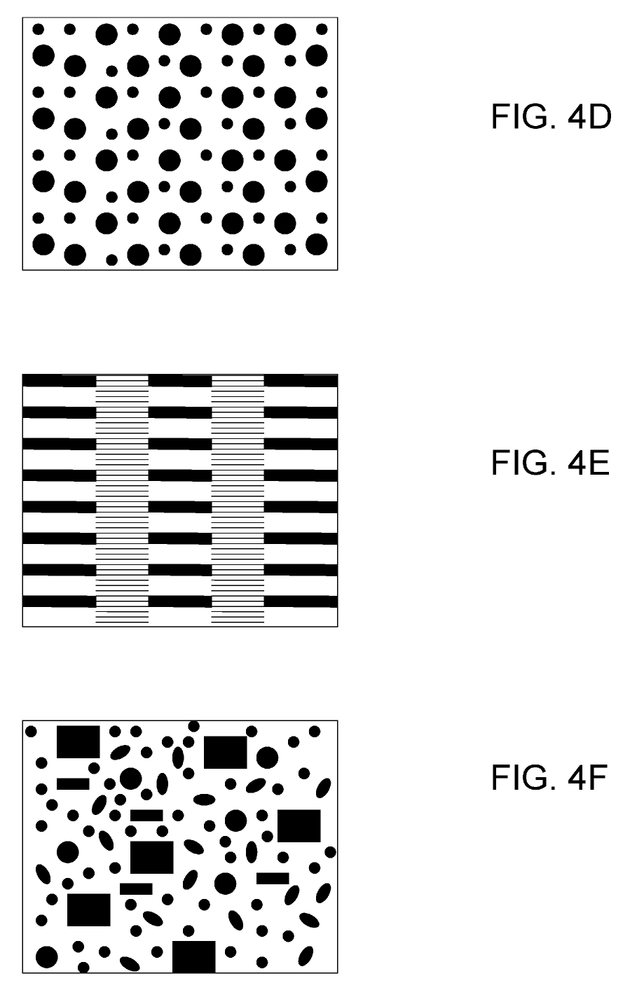

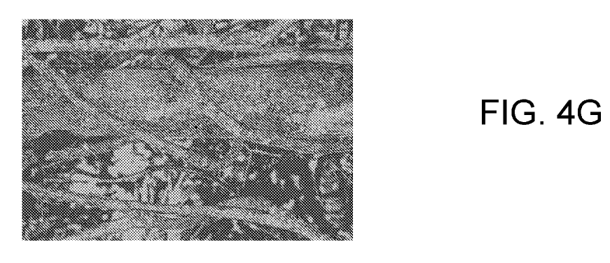

FIGS. 4A-4G illustrate a few examples of patterns that could be used. Each of these patterns is designed to be joined at the top and the bottom to form a closed 3 dimensional loop. The patterns could be implemented on or in the moving element 100, for example by printing in black and white, or gray scale, or color and/or by physically cutting the patterns into the moving element 100. For example, the shaded areas could be cut to a particular depth in the moving element 100 while the white areas remain uncut. For example, the pattern of FIG. 4A can be formed by knurling a small metal or plastic roller. The optical contrast between cut and uncut regions could be imaged by the imaging sensor 130. Such a pattern may comprise regularly spaced elements as shown, for example, in FIGS. 4A-4E, or irregularly spaced elements, as shown, for example, in FIG. 4F.

If the pattern includes regularly spaced elements, it is preferable to include patterns with at least two different spacings, for example the combination of closely spaced and coarsely spaced features can be used to obtain fine resolution in optimal conditions and adequate coarse resolution in suboptimal conditions, such as when gel is applied to the skin. Examples of multiple regularly spaced patterns are shown in FIGS. 4D and 4E. An irregular pattern has similar benefits. An irregular pattern also has the advantage of being less likely to confuse image comparison software or firmware that compares sequential images. It is preferable that the patterned image comprise at least two different elements to further enhance the ability of the system to detect and resolve fine and coarse motion. The patterned image may be a series of lines or dots, or it may be more complicated, such as a picture, as shown in FIG. 4G. The patterned image may also be located within the moving element in a location that is visible to the imaging sensor 130. The patterned image may be printed, attached, etched, molded, or otherwise imprinted onto or into the moving element. Other methods for creating an imagable pattern on the surface are deemed to be within the scope of the invention.

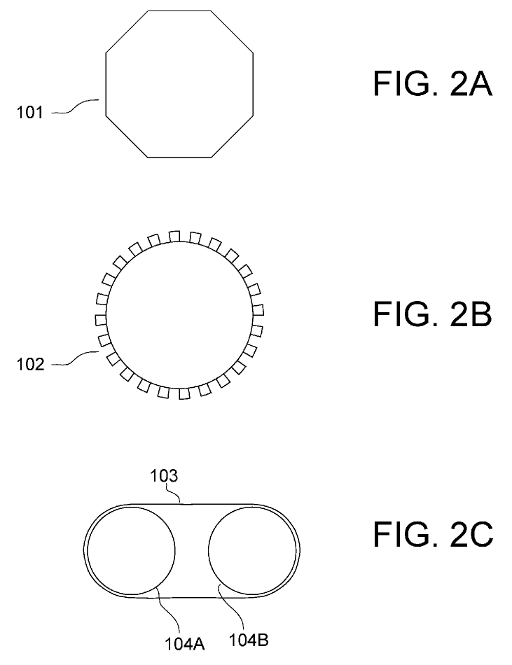

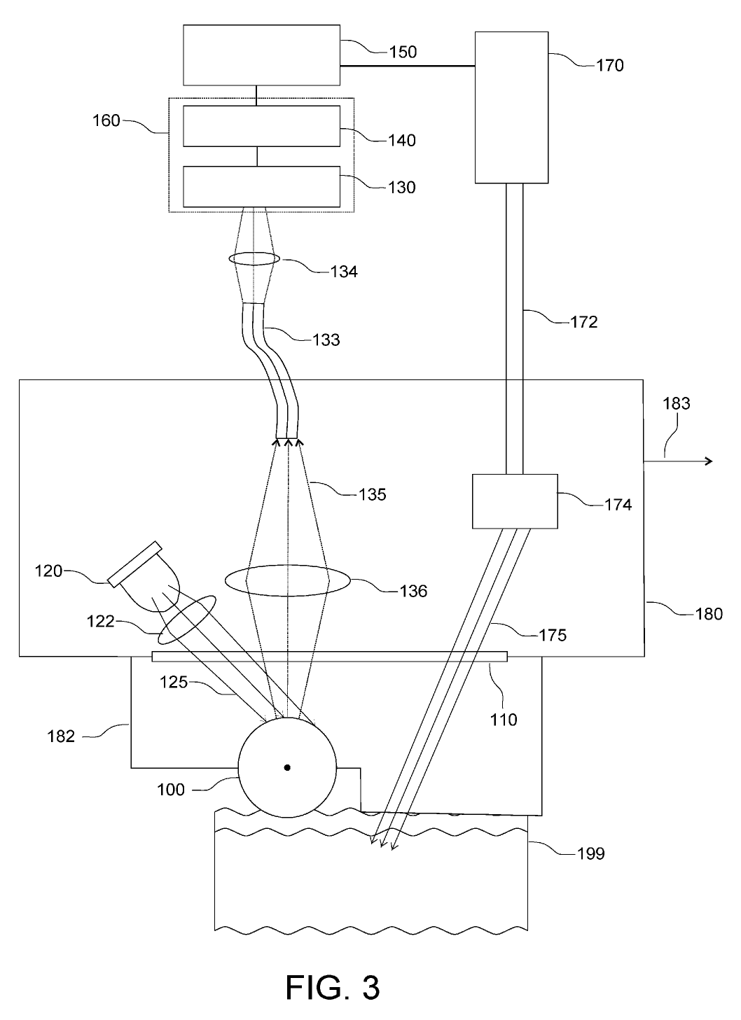

The shape of the moving element 100 can be chosen based on system design constraints. In some embodiments, the moving element is a cylinder, which permits easy rotation and makes it easy to print or attach a patterned image. The moving element can be spherical, which rotates easily and provides a reduced contact area to the skin in comparison to a cylindrical moving element, which can be useful in situations where the skin is sensitive due to treatment. A spherically shaped moving element could beneficially be used in an embodiment that measures motion in two perpendicular dimensions. Other shapes can be used to enhance the friction with the skin to allow more robust measurement of movement. For example, the shapes shown in FIG. 2A-2C each increase the friction with the skin. FIG. 2A illustrates a polygon-shaped cylinder (i.e. a cylinder with a polygonal cross section) that can be used. The flat surfaces of the polygon-shaped cylinder allow easy application of a pattern. FIG. 2B illustrates a geared or toothed shape that could provide better traction on the skin and provide a pattern that is easily imaged. Rounded or pointed spikes or other shapes could alternately be used as desired. FIG. 2C illustrates a belt 103 that is suspended between two rotating members 104A,B. The belt 103 provides a large contact area with the skin surface and thus can provide better traction than geometries with smaller contact area with the skin 199. For each of the above described shapes and for other shapes, the moving element 100 may further comprise a textured surface, or tread, that can further enhance the traction on the skin 199.

The imaging light 135 can be, for example, scattered, diffracted, emitted, fluoresced, or reflected from the moving element 100. The illumination source 120 is optional. Ambient light may be sufficient for the imaging sensor 130 to image the moving element 100. In other implementations, the moving element 100 may fluoresce or emit light that can be detected by the imaging sensor 130.

The imaging sensor 130 may be a charge coupled detector (CCD) chip, a CMOS detector array, or an array of coordinated photodetector cells. Preferably, the imaging sensor 130 has at least 5×5 or, more preferably, at least 15×15 detector elements. The number of detectors is chosen to have adequate resolution for detecting changes in position along a desired direction 183 of handpiece motion.

In some embodiments, the treatment energy source 170 is a light source and part of the treatment energy 175 may be used to replace the illumination source 120. This may occur through appropriate placement of the treatment energy source 170 or by splitting off a portion of the treatment energy 175 using, for example, a beamsplitter (not shown).

Treatment energy source 170 may be located inside or outside the handpiece. The treatment energy source 170 may comprise one or more of lasers, flashlamps, ultrasonic transmitters, monopolar and bipolar RF sources, and RF plasma systems.

The removable tip 182 is configured to allow treatment energy from the treatment energy source 170 to be directed to the target area of skin. For this purpose, the removable tip 182 may comprise, for example, transparent, conductive, or hollow regions that allow treatment energy from energy source 170 to be directed to the target area. One advantage to making the moving element 100 be part of the removable tip 182 is that the moving element can be replaced easily if it gets gummed up or infected with bacteria during treatment due to contact with the patient. Another advantage of making the moving element 100 be part of the removable tip 182 is that different moving elements 100 can be easily swapped out to adjust for different conditions, such as for example to use different patterns on the moving element for different areas of the body or to use differently shaped or sized moving elements for different areas of the body. A set of tips with different moving elements can be provided to allow the same base handpiece to be used to treat different areas of the body and/or to effect different treatments.

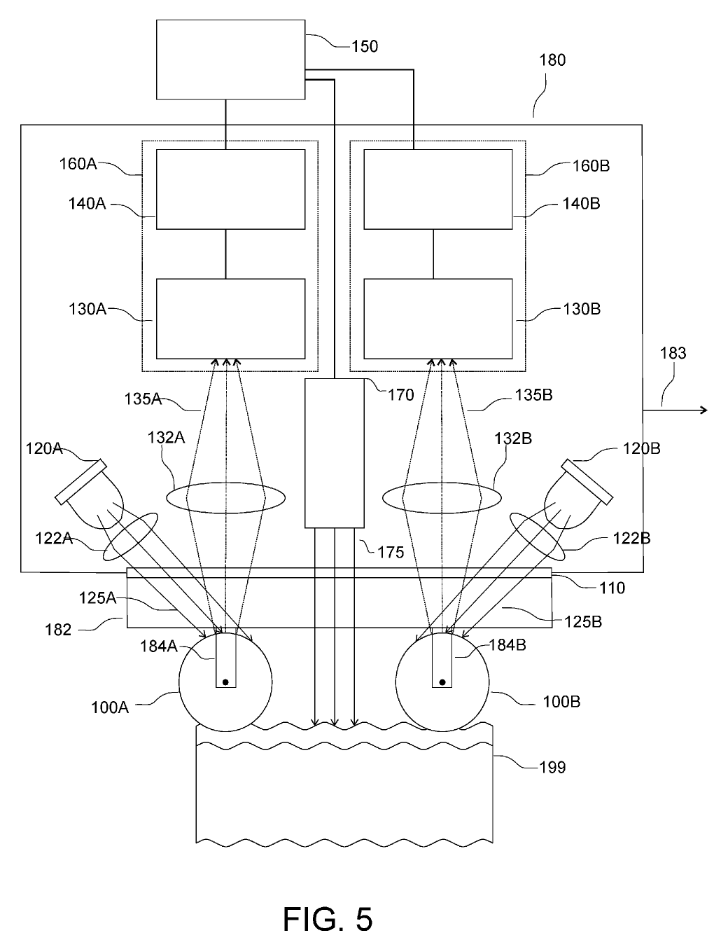

FIG. 5 illustrates another embodiment of the invention that employs two moving elements 100A,B. Having two or more moving elements makes it easier for the handpiece to move across the skin 199. Also in this embodiment, the moving elements 100A,B are supported by forks 184A,B that allow easy removal of the moving element and allow the moving element to be manually replaceable within a tip 182.

The embodiment of FIG. 5 also comprises an additional optical imaging system, (comprising elements 130, 140, 160, 132, 120, and 122) with the additional imaging system components designated by the letters A & B. These components correspond to their numbered counterparts in FIG. 1. Having a second set of optical imaging components improves the reliability of the velocity detection by providing a secondary signal, although it is not necessary to have separate optical imaging systems. In alternate embodiments, some or all of the elements in the optical imaging system may be shared. Signals can be separated using time multiplexing or other separation techniques.

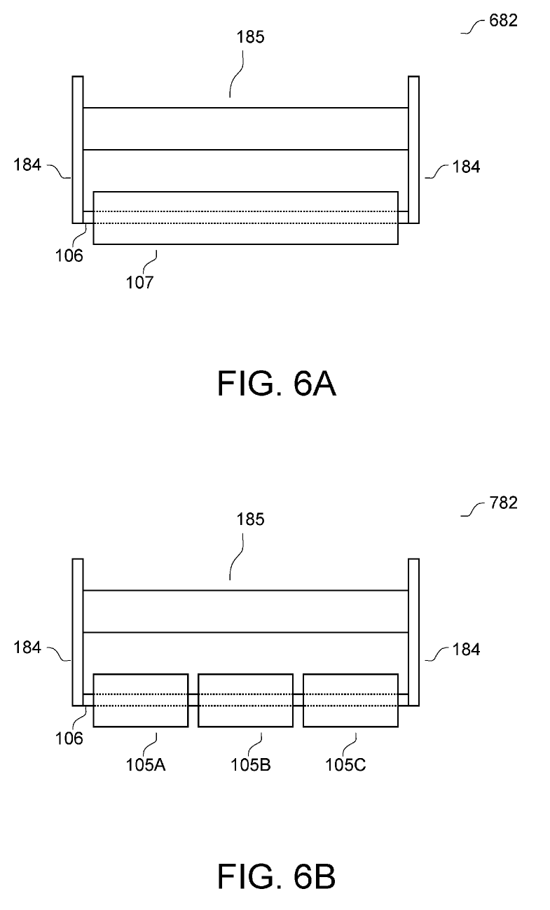

As shown in FIGS. 6A and 6B, the moving element 100 (or 100A,B) may be implemented as a single-piece moving element 107 or may be implemented as a segmented moving element 105A,B,C. In either configuration, moving element is supported by an axis 106. The single-piece moving element 107 of FIG. 6A has the advantage of being easier to manufacture. The segmented moving element 105A,B,C allows the handpiece to better conform to convex or concave curvatures of the skin 199, which is useful for improving tracking across narrow bony contours of the skin 199. The segmented moving element 105 can comprise one or more pieces. The segmented moving element 105 shown in FIG. 6B comprises three separate segments FIG. 6B, but the separate segments 105A,B,C may be joined together using weaker materials or flexible struts (not pictured) that preserve the integrity of the roller as a single unit, but which still allow the segmented moving element 105 to conform to contours of the skin 199 better than if the moving element were a single, uniform piece. The axis 106 may be made of flexible material to allow better conformity.

FIG. 3 describes an alternate embodiment of the invention that further comprises image relaying optics 133 to allow positioning of the imaging sensor 130 at a location outside of the handpiece 180. At least a portion of moving element 100 is imaged by imaging lens 136 into the input of the image relaying optics 133. A second imaging lens 134 can then be used to image the output of the image relaying optics 133 onto the imaging sensor 130. This embodiment can further comprise a scanner 174 and a waveguide 172. The waveguide 172 is optional and directs treatment energy 176 from the treatment energy source 170 to the scanner 174. The scanner 174 can deflect the treatment energy to different desired locations on the skin as the handpiece is moved across the skin.

In some embodiments, the imaging sensor 130 and sensor analysis processor 140 may be combined into a single package or a single chip. One example of such a combination is an optical mouse chip 160 from Avago Technologies, Inc. (e.g. part number ADNS-3080) as schematically illustrated in FIGS. 1 and 3. In the Avago Technologies ADNS-3080, the imaging sensor 130 is a CMOS detector array.

Waveguide 172 can be an optical waveguide, an RF waveguide, or an acoustical waveguide, depending on the selected treatment energy source 170. In selected embodiments, waveguide 172 can be a single-mode or multimode optical fiber or an articulating arm.

Scanner 174 can be a galvanometer based optical scanner or other scanner designed to scan optical, acoustic, or RF energy. Several examples of applicable scanners 174 are well known in the art. In one embodiment, scanner 174 is a reflective optical scanner as described in copending U.S. patent application Ser. No. 11/158,907 entitled “Optical pattern generator using a single rotating component,” which is herein incorporated by reference.

In some embodiments, the inventive apparatus can be used to create a discrete pattern of treatment zones at the target tissue and thus spare portions of the target tissue between treatment zones such that rapid healing occurs in individual treatment zones. In some embodiments, a scanner 174 can be beneficially used to create discrete treatment zones in the target region of skin. In a preferred embodiment, the scanner 174 can be used to create individual treatment zones are less than 1 mm wide at the narrowest dimension. In other embodiments, the scanner is optional and discrete treatment zones can be created by employing patterning element (not shown) in the removable tip 182. The patterning element can be chosen based on the type of treatment energy source 170 and other system constraints. The patterning element may be, for example, a patterned mask, a microlens array, an array of focusing elements, a waveguide array, a patterned electrode, or any combination of such elements. Additional benefits of discrete treatment zones and other embodiments that may be used to create discrete treatment zones are disclosed in co-pending U.S. patent applications Ser. Nos. 10/367,582 (filed Feb. 14, 2003 and entitled “Method and apparatus for treating skin using patterns of optical energy”), 10/888,356 (filed Jul. 9, 2004 and entitled “Method and Apparatus for fractional photo therapy of skin”), and 60/773,192 (filed Feb. 13, 2006 and entitled “Laser system for treatment of skin laxity”), each of which is herein incorporated by reference.

A scanner 174 can be used in other embodiments to create uniform treatment over the entire treatment area. Using an optical scanner could, for example, allow the use of a low power laser as the treatment energy source 170 to treat a large target region uniformly without having to move the handpiece between laser treatment pulses.

In some embodiments, the image relaying optics 133 is a fiber array. Fiber arrays are available from Nanoptics, Inc. (Gainesville, Fla.). Other waveguide arrays could also be used in place of the fiber array. In another embodiment, the image relaying optics 133 can be a series of lenses that relays the image from the input of the image relaying optics 133 to the output with a magnification that can be chosen as desired. In another embodiment, the image relaying optics 133 is an array of flexible, internally reflective, hollow tubes. Preferably, the number of optical waveguides or number of optical fibers in the fiber array bundle is at least two times, and more preferably at least five times, the number of discrete detector elements in the imaging sensor 130 in order to have optimal image quality for the imaging sensor 130.

One advantage of the embodiment described in FIG. 3 is that it allows the handpiece to be smaller than the embodiment described in FIG. 1 due to the remote location of some of the larger and bulkier components, such as the treatment energy source 170 and the optical mouse chip 160. For example, these components can be placed in a system chassis that is remotely located from the handpiece and can be separated by a distance of at least 50 cm or at least 2 meters.

The system may further comprise a sponge that is wetted with alcohol or other solvent to remove gel or other material that contaminates the roller during use. This sponge may be attached to the tip or may be sold separately. The solvent may be released automatically from an attached chamber or may be applied by the user. Other solutions for cleaning the tip, such as a wiper that wipes the roller surface, are also considered to be within the scope of the invention.

Although the detailed description contains many specifics, these should not be construed as limiting the scope of the invention but merely as illustrating different examples and aspects of the invention. It should be appreciated that the scope of the invention includes other embodiments not discussed in detail above. For example, the moving element 100 is drawn as part of the removable tip 182, but those skilled in the art will recognize that the moving element may be incorporated into other portions of the handpiece. The aspects of this invention as described above can be further combined to create other embodiments that are within the scope of this invention. For example, each of the components including but not limited to the image relaying optics 133, the waveguide 172, the optical scanner 174, and each of the elements described in FIGS. 2A-2C could be used in the embodiment described in FIG. 1, either individually or in any combination with one or more of the other components. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

In the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

What is claimed is:

1. An apparatus for dermatological treatment comprising:
a handpiece configured to receive energy from an energy source, wherein the handpiece delivers said energy to a target region of skin for dermatological treatment and the handpiece is moved across the skin during treatment;
a moving element that contacts the skin and moves in response to the motion of the handpiece across the skin;
an imaging sensor that captures at least two images of at least a portion of the moving element;
a processor that compares at least two of the captured images to determine at least one positional parameter of the moving element; and
a controller that adjusts a parameter of the energy source to alter the dermatological treatment in response to the determination.

2. The apparatus of claim 1, wherein the energy comprises at least one of electromagnetic energy and ultrasonic energy.

3. The apparatus of claim 2, wherein the energy source comprises a laser source.

4. The apparatus of claim 2, wherein the energy source comprises a radio-frequency source.

5. The apparatus of claim 2, wherein the energy source comprises a radio-frequency source that delivers energy to plasma for treatment of the skin.

6. The apparatus of claim 2, wherein the comparison of the captured images distinguishes between motion of the moving element in two opposite directions relative to the orientation of the handpiece.

7. The apparatus of claim 2, wherein the comparison of the captured images distinguishes between motion of the moving element in two perpendicular directions relative to the orientation of the handpiece.

8. The apparatus of claim 2, further comprising:
image relaying optics that form part of an optical path between at least a portion of the moving element and the imaging sensor.

9. The apparatus of claim 8, wherein said image relaying optics comprise a fiber array.

10. The apparatus of claim 8, wherein said image relaying optics comprise an array of optical waveguides and the number of waveguides in the array is at least twice the number of individual detector elements in the imaging sensor.

11. The apparatus of claim 2, wherein the controller automatically triggers the energy source in response to a measured movement of the moving element of a predetermined distance.

12. The apparatus of claim 2, wherein the controller adjusts the firing rate of the energy source in response to the determined positional parameter.

13. The apparatus of claim 2, wherein the controller adjusts the power level of the energy source in response to a change in at least one of speed and velocity of the moving element.

14. The apparatus of claim 13, wherein the energy source is a laser operating in continuous wave mode.

15. The apparatus of claim 13, wherein the energy source is a laser operating in pulsed mode.

16. The apparatus of claim 2, wherein the controller adjusts a pulse repetition rate in response to the determined positional parameter.

17. The apparatus of claim 2, wherein the controller adjusts an energy dose in response to the determined positional parameter.

18. The apparatus of claim 2, wherein the imaging sensor comprises a CCD chip.

19. The apparatus of claim 18, wherein the imaging sensor comprises a CCD chip that is separated from the handpiece by at least 50 centimeters.

20. The apparatus of claim 2, wherein the imaging sensor comprises a CMOS detector array.

21. The apparatus of claim 20, wherein the imaging sensor comprises a CMOS detector array that is separated from the handpiece by at least 50 centimeters.

22. The apparatus of claim 2, wherein the moving element comprises a rotating element.

23. The apparatus of claim 2, wherein the moving element is substantially spherical in shape.

24. The apparatus of claim 2, wherein the moving element is substantially a round or polygonal cylinder in shape.

25. The apparatus of claim 2, wherein the moving element comprises a moving band that contacts the skin.

26. The apparatus of claim 2, wherein the moving element comprises a textured surface that enhances traction of the moving element on the skin.

27. The apparatus of claim 2, wherein the moving element is not round.

28. The apparatus of claim 2, wherein the moving element comprises a patterned image.

29. The apparatus of claim 28, wherein the patterned image is non-repeating.

30. The apparatus of claim 28, wherein the patterned image comprises at least two different elements or two different spacings between adjacent elements.

31. The apparatus of claim 2, wherein the at least two images are captured within 50 milliseconds.

32. The apparatus of claim 31, wherein the at least two images are captured within 5 milliseconds.

33. The apparatus of claim 2, further comprising a scanner that directs energy from the treatment energy source to different portions of the target region.

34. The apparatus of claim 2, further comprising a patterning element that directs energy from the treatment energy source to multiple discrete portions of the target region.

35. The apparatus of claim 2, wherein discrete treatment zones are created in the target region of skin.

36. The apparatus of claim 2, wherein the moving element is a single piece.

37. The apparatus of claim 2, wherein the moving element is segmented.

38. The apparatus of claim 2, further comprising a second moving element that contacts the skin and moves in response to the motion of the handpiece across the skin.

39. A method of dermatological treatment comprising the steps of
directing energy from a treatment energy source to a target region of skin using a handpiece;
manually moving the handpiece across the target region to cause a moving element attached to the handpiece and in contact with the skin to move relative to an imaging sensor;
capturing at least two images of at least a portion of the moving element;
determining at least one relative positional parameter of the moving element by comparing the captured images; and
automatically adjusting a parameter of the energy source to alter the dermatological treatment in response to the determined positional parameter.

40. The method of claim 39, wherein the dermatological treatment is a cosmetic dermatological treatment.

41. The method of claim 39, wherein the dermatological treatment is a noninvasive cosmetic dermatological treatment.

42. The method of claim 39, wherein the step of determining relative positional parameter distinguishes between motions of the moving element in two opposite directions.

43. The method of claim 39, wherein the step of capturing at least two images of at least a portion of the moving element comprises relaying said images from the moving element via a fiber array to the imaging sensor.

44. The method of claim 39, wherein the step of directing energy to a target region of skin comprises scanning the energy to different portions of the target region of skin.

45. The method of claim 39, wherein the step of directing energy to a target region of skin comprises creating discrete treatment zones in the target region of skin.
